(19) United States
(12) Patent Application Publication
(10) Pub. No.: US 2015/0196321 A1
(43) Pub. Date: Jul. 16, 2015

(54) CANNULA

(71) Applicants: BIVACOR PTY LTD, Wilston (AU); QUEENSLAND UNIVERSITY OF TECHNOLOGY, Brisbane (AU)

(72) Inventors: Shaun GREGORY, Bracken Ridge (AU); Armin LIEBHARDT, Wivenhoe Pocket (AU); Mark PEARCY, Capalaba (AU); Daniel TIMMS, Wilston (AU)

(21) Appl. No.: 14/596,091

(22) Filed: Jan. 13, 2015

(30) Foreign Application Priority Data
Jan. 14, 2014 (AU) 2014900108

(51) Int. Cl. A61B 17/34; A61B 17/32; A61B 17/3205; A61M 1/10; A61B 17/04

(52) U.S. Cl. CPC A61B 17/3423; A61M 1/1008; A61B 17/0401; A61B 17/32053; A61B 17/320016; A61B 17/3478; A61B 2017/3425; A61B 2017/3429; A61B 2017/347; A61M 2205/0222; A61M 2210/125

(57) ABSTRACT

A cannula including a hollow cannula body having first and second tubular end portions, a collapsible section interconnecting the end portions, the collapsible section including a plurality of circumferentially spaced arms extending between the end portions, wherein in an extended configuration the arms are substantially aligned with the first and second end portions and in a collapsed configuration the arms deform to extend radially outwardly and a flange extending radially outwardly from the first end portion, so that the arms and flange are spaced apart when the cannula body is in the collapsed configuration, thereby allowing tissue to be sandwiched therebetween to thereby effect a seal between the cannula and the tissue so that the cannula provides an opening through the tissue.

### CROSS REFERENCE TO RELATED APPLICATIONS

This application claims priority to Australian Provisional Patent Application No. 2014900108, filed Jan. 14, 2014 and titled “CANNULA,” which is herein incorporated by reference in its entirety.

### INCORPORATION BY REFERENCE

All publications and patent applications mentioned in this specification are herein incorporated by reference to the same extent as if each individual publication or patent application was specifically and individually indicated to be incorporated by reference.

### FIELD

The present invention relates to a cannula and a method of inserting a cannula and in one particular example to inserting a cannula through tissue in a biological subject.

### BACKGROUND

The reference in this specification to any prior publication (or information derived from it), or to any matter which is known, is not, and should not be taken as an acknowledgment or admission or any form of suggestion that the prior publication (or information derived from it) or known matter forms part of the common general knowledge in the field of endeavour to which this specification relates.

During surgical procedures it is often necessary to insert a connector, such as a cannula, into an organ of a biological subject. For example, this might be required in the event that a heart pump is to be fitted to a patient's heart for use as a ventricular assist device. Traditional techniques have involved the use of sutures, to connect and retain cannulae in position. However, such techniques are time consuming, difficult to perform and often difficult to reverse, should there be a need to remove and replace the cannula after installation.

U.S. Pat. No. 7,931,581 describes surgical tools for off-pump surgery. In an embodiment, a surgical connector for coupling an organ to a medical device comprises an inner body. The surgical connector further comprises an outer hollow body slidably disposed around said inner body. Moreover, the connector comprises a distal sealing member disposed around said inner body. The distal sealing member has a collapsed position and an expanded position. The distal sealing member is also capable of being locked in said expanded position. The surgical connector further comprises a proximal sealing member disposed around said outer hollow body, said proximal sealing member is proximal to said distal sealing member. The disclosed surgical connector employs a novel press-fit mechanism. The press-fit mechanism allows a surgeon to attach the connector to an organ without the use of sutures. The surgical connector also incorporates an expandable sealing member that can be locked into the expanded position.

However, the arrangements suffer from a number of disadvantages. Firstly, the arrangements require separate steps for coring the organ and then subsequently inserting the connector, making the process difficult to perform during a surgical procedure, particularly without undue fluid loss between coring of the organ and insertion of the connector. Additionally, the arrangement of the apparatus is quite complex, making it expensive to manufacture and difficult to use.

### SUMMARY OF THE DISCLOSURE

The present invention seeks to ameliorate one or more of the problems associated with the prior art.

In a first broad form the present invention seeks to provide a cannula including a hollow cannula body having: (a) first and second tubular end portions; (b) a collapsible section interconnecting the end portions, the collapsible section including a plurality of circumferentially spaced arms extending between the end portions, wherein in an extended configuration the arms are substantially aligned with the first and second end portions and in a collapsed configuration the arms deform to extend radially outwardly; (c) a flange extending radially outwardly from the first end portion, so that the arms and flange are spaced apart when the cannula body is in the collapsed configuration, thereby allowing tissue to be sandwiched therebetween to thereby effect a seal between the cannula and the tissue so that the cannula provides an opening through the tissue.

Typically the arms include deformable portions that cause the arms to deform in predefined positions when collapsing.

Typically in the collapsed configuration, the arms extend outwardly in a generally “V” shaped arrangement.

Typically the arms include a first arm portion coupled to the first end portion and a second arm portion coupled to the second end portion, the first and second arm portions being interconnected and wherein the first arm portion is shorter than the second arm portion so that the first arm portion is substantially parallel to the flange when the cannula body is in the collapsed configuration.

Typically the cannula includes a locking mechanism for locking the cannula in the collapsed configuration.

Typically the locking mechanism is adjustable to allow the relative separation of the flange and arms to be adjusted.

Typically the locking mechanism interconnects the first and second end portions.

Typically the cannula includes a tubular body extending through and coaxially inwardly of the cannula body.

Typically the tubular body includes a shoulder at a first end for engaging the second end portion.

Typically the tubular body includes a number of axially spaced body teeth extending circumferentially around at least part of an outer surface of the tubular body and wherein the first end portion includes a number of axially spaced cannula teeth extending circumferentially around at least part of an inner surface of the first end portion, the body teeth and cannula teeth selectively engaging to thereby lock the tubular body relative to the first end portion.

Typically relative rotation of the tubular body and first end portion allow the body teeth and cannula teeth to selectively disengage.

Typically the cannula includes a sleeve extending over and coaxially outwardly of at least part of the cannula body.

Typically the sleeve is a silicone sleeve.

Typically the sleeve has a low friction outer surface to facilitate insertion of the cannula into the tissue.

Typically the sleeve includes a roughened outer surface at least proximate an end of the sleeve adjacent the flange.

Typically the flange includes a felt surface that in use engages the tissue surface to thereby seal the cannula against the tissue.

In a second broad form the present invention seeks to provide a cannula system including: a) a cannula including a hollow cannula body having: i) first and second tubular end portions; ii) a collapsible section interconnecting the end portions, the collapsible section including a plurality of circumferentially spaced arms extending between the end portions, wherein in an extended configuration the arms are substantially aligned with the first and second end portions and in a collapsed configuration the arms deform to extend radially outwardly; iii) a flange extending radially outwardly from the first end portion, so that the arms and flange are spaced apart when the cannula body is in the collapsed configuration, thereby allowing tissue to be sandwiched therebetween to thereby effect a seal between the cannula and the tissue so that the cannula provides an opening through the tissue; and, b) a corer including: i) a corer body that in use extends through and coaxially inwardly of the cannula body; ii) a handle proximate a first end of the corer body for manipulating the corer; iii) a corer cutting edge at a second opposing end of the corer body for coring the tissue to thereby creating an opening in the tissue; and, iv) an engagement mechanism for selectively engaging the cannula, thereby allowing the cannula body to be collapsed in use.

Typically the cannula includes a tubular body extending through and coaxially inwardly of the cannula body, the corer extending through and coaxially inwardly of the tubular body.

Typically the engagement mechanism includes a corer shoulder that engages the tubular body.

Typically the engagement mechanism includes a corer threaded portion that selectively couples to a corresponding tubular body threaded portion upon relative rotation of the corer and tubular body.

Typically the corer threaded portion is provided on an outer surface of the corer body and the tubular body threaded portion is provided on an inner surface of the tubular body.

Typically the corer includes first and second coaxially arranged corer bodies, and wherein relative axial movement of the corer bodies urges fingers of the second corer body outwardly to thereby engage the tubular body.

Typically the fingers define corer cutting edges.

Typically the first and second corer bodies are threadingly engaged to cause relative axial movement of the corer bodies.

Typically the corer includes at least one of: a) a hook that in use is coupled to a suture passing through the tissue to be removed; and, b) a tissue screw that in use engages the tissue to be removed.

In a third broad form the present invention seeks to provide a method of inserting a cannula mounted on a corer, wherein: a) the cannula includes a cannula body having: i) first and second tubular end portions; ii) a collapsible section interconnecting the end portions, the collapsible section including arms extending between the end portions; iii) a flange extending radially outwardly from the first end portion; and, b) the corer includes: i) a corer body that in use extends through and coaxially inwardly of the cannula body; ii) a handle for manipulating the corer; iii) a corer cutting edge; and, iv) an engagement mechanism, wherein the method includes: (1) inserting the corer cutting edge through the tissue to thereby create an opening in the tissue; (2) urging the cannula through the opening in the tissue, with the cannula in an extended configuration in which the arms are substantially aligned with the first and second end portions; (3) urging the corer towards the first end portion so that the engagement mechanism urges the second end portion towards the first end portion to thereby move the cannula to a collapsed configuration in which the arms deform to extend radially outwardly so that the arms and flange are spaced apart thereby allowing tissue to be sandwiched therebetween to thereby secure the cannula to the tissue; and, (4) withdrawing the corer from the cannula so that the cannula provides an opening through the tissue.

Typically the method includes using a locking mechanism to lock the cannula in the collapsed configuration.

Typically the cannula includes a tubular body extending through and coaxially inwardly of the cannula body and wherein the method includes rotating a tubular body relative to the cannula body to thereby selectively engage the locking mechanism.

Typically the cannula includes a tubular body extending through and coaxially inwardly of the cannula body, wherein the engagement mechanism includes a corer threaded portion that selectively couples to a corresponding tubular body threaded portion and wherein the method includes rotating the corer relative to the tubular body thereby allowing the corer to be withdrawn from the cannula.

### BRIEF DESCRIPTION OF THE DRAWINGS

An example of the present invention will now be described with reference to the accompanying drawings, in which:—

FIG. 1A is a schematic perspective view of an example of a cannula body in an extended configuration;

FIG. 1B is a schematic side view of the cannula body of FIG. 1A;

FIG. 1C is a schematic perspective view of the cannula body of FIG. 1A in a collapsed configuration;

FIG. 1D is a schematic side view of the cannula body of FIG. 1A in the collapsed configuration;

FIG. 2A is a schematic perspective view of an example of a tubular body;

FIG. 2B is schematic side view of the tubular body of FIG. 2A;

FIG. 2C is a schematic end view of the tubular body of FIG. 2A;

FIG. 2D is a schematic end view of the cannula body of FIG. 1A;

FIG. 2E is a schematic rear perspective view of the cannula body of FIG. 1A;

FIG. 3A is a schematic perspective view of an example of a sleeve;

FIG. 3B is a schematic side view of the sleeve of FIG. 3A;

FIG. 3C is a schematic perspective view of the sleeve of FIG. 3A in a collapsed configuration;

FIG. 3D is a schematic side view of the sleeve of FIG. 3C in the collapsed configuration;

FIG. 4A is a schematic cutaway perspective view of an example of a cannula including the cannula body of FIG. 1A, the tubular body of FIG. 2A and the sleeve of FIG. 3A;

FIG. 4B is a schematic side cutaway view of the cannula of FIG. 4A;

FIG. 4C is a schematic cutaway perspective view of the cannula of FIG. 4A in a collapsed configuration;

FIG. 4D is a schematic cutaway side view of the cannula of FIG. 4A in the collapsed configuration;

FIG. 5A is a schematic perspective view of an example of a corer;

FIG. 5B is a schematic side view of the corer of FIG. 5A;

FIG. 5C is a schematic perspective cutaway view of an example of the cannula of FIG. 4A including the corer of FIG. 5A inserted therein;

FIG. 5D is a schematic side cutaway view of the cannula and corer of FIG. 5C;

FIG. 6A is a schematic side view of an example of a cannula and corer prior to insertion;

FIG. 6B is a schematic side view of the cannula and corer of FIG. 6A upon insertion;

FIG. 6C is a schematic side view of the cannula of FIG. 6A installed;

FIG. 7A is a schematic perspective view of a second example of a corer;

FIG. 7B is a schematic perspective view of a second example of a tubular body;

FIG. 7C is a schematic cut-away view of a cannula including the corer of FIG. 7A and tubular body of FIG. 7B;

FIG. 7D is a schematic perspective view of a cannula in including the tubular body of FIG. 7B in a collapsed position;

FIG. 7E is a schematic perspective cut-away view of a cannula of FIG. 7D;

FIG. 8A is a schematic perspective view of a third example of a corer;

FIG. 8B is a schematic perspective view of a third example of a tubular body;

FIG. 8C is a schematic cut-away view of a cannula including the corer of FIG. 8A and the tubular body of FIG. 8B;

FIG. 8D is a schematic perspective view of a cannula in including the tubular body of FIG. 8B in a collapsed position;

FIG. 8E is a schematic perspective cut-away view of a cannula of FIG. 8D;

FIG. 9A is a schematic perspective view of a second example of a corer;

FIG. 9B is a schematic side view of the corer of FIG. 9A;

FIG. 9C is a schematic perspective view of a first corer body of the corer of FIG. 9A;

FIG. 9D is a schematic side view of the first corer body of FIG. 9C;

FIG. 9E is a schematic perspective view of a second corer body of the corer of FIG. 9A;

FIG. 9F is a schematic side view of the second corer body of FIG. 9E;

FIG. 9G is a schematic perspective cut away view of the corer of FIG. 9A;

FIG. 9H is a schematic side cut away view of the corer of FIG. 9A;

FIG. 10A is a schematic perspective view of a third example of a corer;

FIG. 10B is a schematic perspective cut away view of the corer of FIG. 10A; and,

FIG. 10C is a schematic perspective exploded view of the corer of FIG. 10A.

### DETAILED DESCRIPTION

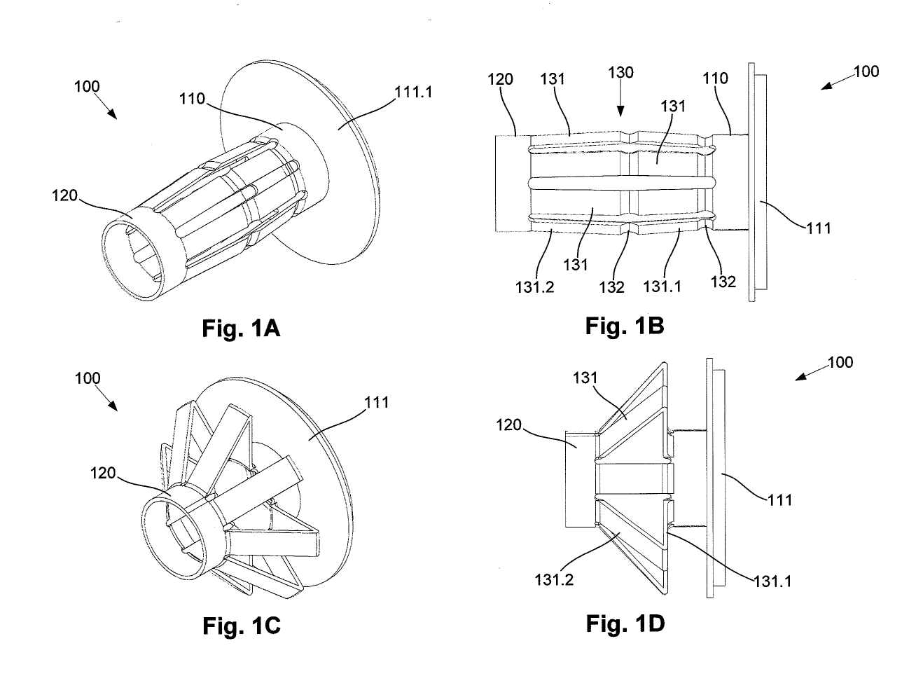

A first example of a cannula will now be described with reference to FIGS. 1A to 1D.

In this example, the cannula includes a hollow cannula body 100 including first and second tubular end portions 110, 120, interconnected via a collapsible section 130. The collapsible section 130 includes a number of circumferentially spaced arms 131 extending between the first and second end portions 110, 120. In an extended configuration, shown in FIGS. 1A and 1B, the arms 131 are substantially aligned with the first and second end portions 110, 120, whereas in a collapsed configuration shown in FIGS. 1C and 1D the arms deform to extend outwardly from the cannula body 100.

The first end portion 110 includes a flange 111 extending radially outwardly from the first end portion 110. In use, the arms 131 and flange 111 are spaced apart when the cannula body is in the collapsed configuration, as shown in FIG. 1D, thereby allowing tissue, such as a wall of an organ, to be sandwiched therebetween to thereby secure the cannula in position so that the cannula 100 provides an opening through the tissue.

Thus, in use, the above described cannula can be inserted through tissue, such as an organ wall or the like, whilst the cannula body is in the extended configuration. Once inserted into the organ, the cannula body can be collapsed so that the organ wall is gripped between the arms 131 and the flange 111, thereby securing the cannula in position.

Accordingly, the above described arrangement provides a cannula that can be inserted into and secured to tissue. Furthermore, the cannula is generally simpler than previous arrangements, making it cheaper to manufacture and more reliable in use.

A number of further features will now be described.

The flange 111 may include a felt surface 111.1 facing the arms, so that in use the felt is provided in contact with the tissue. This can assist in protecting the tissue, whilst also helping to provide a seal between the tissue and the flange.

In the example of FIG. 1A, the arms include deformable portions 132, typically formed from cut-outs or thin sections of the arms, which cause the arms to deform in predefined positions when collapsing. This is performed to ensure that the arms extend outwardly in a generally “V” shaped arrangement, with a first arm portion 131.1 coupled to the first end portion 110 being shorter than a second arm portion 131.2 coupled to the second end portion 120, so that the first arm portion 131.1 is substantially parallel to the flange 111, when the cannula body 100 is in the collapsed configuration shown in FIGS. 1C and 1D. This also results in a flared appearance, which can assist in enhancing flow, as well as securing the cannula in place and separating the wound site from the inflow of the cannula. However, this is not essential and alternatively, the arms 131.1, 131.2 can be of equal lengths. Each arm may also be separated and joined via a hinge or concertina like arrangement to promote deformation while reducing the risk of arm fracture.

In one example, the cannula includes a locking mechanism for locking the cannula in the collapsed configuration, thereby preventing the cannula returning to the extended configuration once installed. This can be used to ensure that the cannula is retained in position once correctly inserted into the body. The locking mechanism can take any appropriate form and is typically adjustable to allow the relative separation of the flange 111 and arms 131 to be adjusted, thereby allowing different thicknesses of tissue to be accommodated, as well as allowing for adjustment of the force of compression on the tissue.

The locking mechanism can interconnect the first and second end portions directly. However, alternatively a tubular body can be provided that extends through and coaxially inwardly of the cannula body, with this operating to provide the locking mechanism.

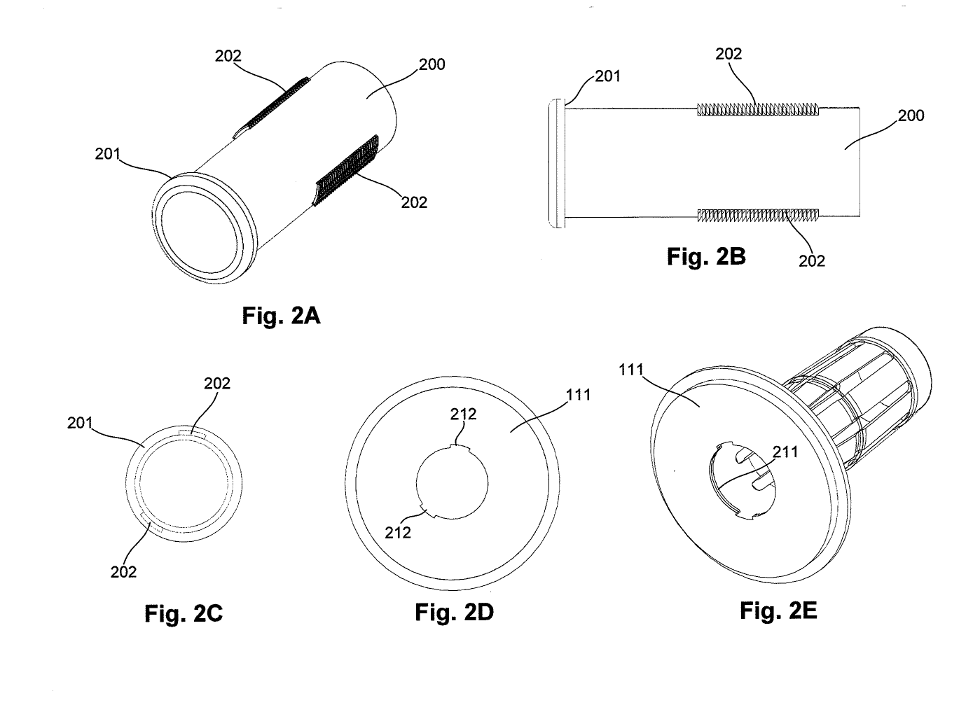

An example of a tubular body will now be described in more detail with reference to FIGS. 2A and 2B.

In this example, the tubular body 200 includes a shoulder 201 provided at a first end, which in use engages the second end portion 120 of the cannula body 100. The tubular body 200 further includes a number of axially spaced body teeth 202, extending circumferentially around at least part of an outer surface of the tubular body 200. In this example, as shown in FIG. 2C, two separate regions of body teeth 202 are provided, with each region being circumferentially spaced around the tubular body 200. In this example,

As shown in FIGS. 2D and 2E, the first end portion 110 includes a number of axially spaced cannula teeth 211, extending circumferentially around at least a part of an inner surface of the first end portion 110. The body teeth 202 and the cannula teeth 211 selectively engage to thereby lock the tubular body 200 relative to the first end portion 110.

It will be appreciated that during this process the shoulder 201 engages the second end portion 120 so that as the tubular body 200 is pulled through the first end portion, this urges the second end portion 120 towards the first end portion 110, thereby causing the cannula body to move into the collapsed configuration. Selective engagement of the body teeth 202 with the first cannula teeth 211 can then be used to lock the first and second end portions relative to each other, thereby retaining the cannula body in a collapsed configuration. Furthermore, by providing the body teeth 202 axially spaced along the tubular body 200, this allows the relative separation of the first and second end portions 110, 120 to be controlled, which in turn adjusts the relative separation of the first arm portions 131.1 and the flange 111.

In this example, it can also be seen that the regions of body teeth 202 can align with respective gaps 212 in the cannula teeth 211, so that the tubular body 200 can be moved relative to the first end portion 110 without the teeth engaging. This allows relative rotation of the tubular body 200 and first end portion 110 to be used to allow the body teeth 202 and cannula teeth 211 to selectively disengage, to initially assemble the two portions, and so that the locking mechanism can be disengaged after compression, for example to adjust the spacing between the first arm portions 131.1 and the flange 111, or to allow the cannula to be returned to the extended configuration for removal. In one example, relative rotation of the tubular body 200 and the first end portion 110 can only be achieved by forcibly overcoming a ridge within the teeth. This ridge would serve to prevent accidental rotation and subsequent return to the extended configuration during normal use. Alternatively, another locking mechanism could be used to prevent relative rotation of the tubular body 200 and the first end portion 110, such as a grub screw or magnet or alike.

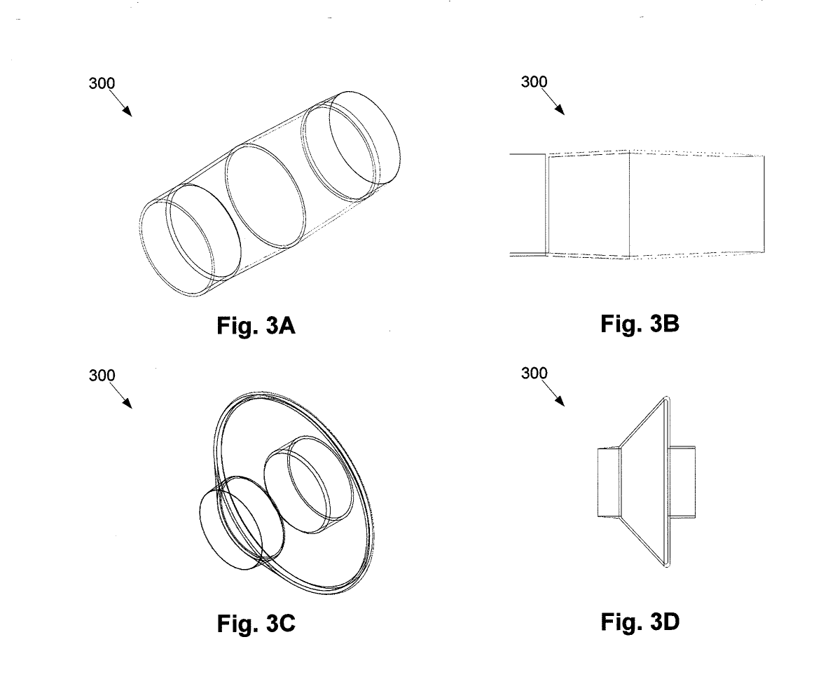

The cannula can also include a sleeve, which can be formed form any suitable material such as silicone or the like, extending over and coaxially outwardly of at least part of the cannula body 100. This can be used to assist with insertion of the cannula into tissue and/or in providing a seal between the tissue and cannula, as well as to assist in preventing blood from stagnating around the collapsing arms. An example of a silicone sleeve will now be described with reference to FIGS. 3A to 3D.

In this example, the silicone sleeve 300 has a generally cylindrical shape that can be positioned over the cannula body and which can accommodate the cannula body in the extended or collapsed configurations as shown for example in FIGS. 3C and 3D.

The silicone sleeve can have a low friction outer surface, for example due to the inherent properties of the silicone or through application of a suitable coating, thereby assisting in insertion of the sleeve and hence cannula into the body.

The sleeve can include a roughened outer surface at least proximate an end of the sleeve near the flange. The roughened surface could be formed by treatment of the sleeve material, coating, or using a woven fabric, or the like. This can be used to assist in promoting tissue regrowth, further enhancing the seal between the tissue and cannula, and allowing a relatively light compressive force, which can be advantageous to counteract tissue becoming fibrous due to sandwiching between the arms and flange. Tissue growth can further be enhanced through impregnation or coating of the sleeve by growth factors, gelatin, or the like.

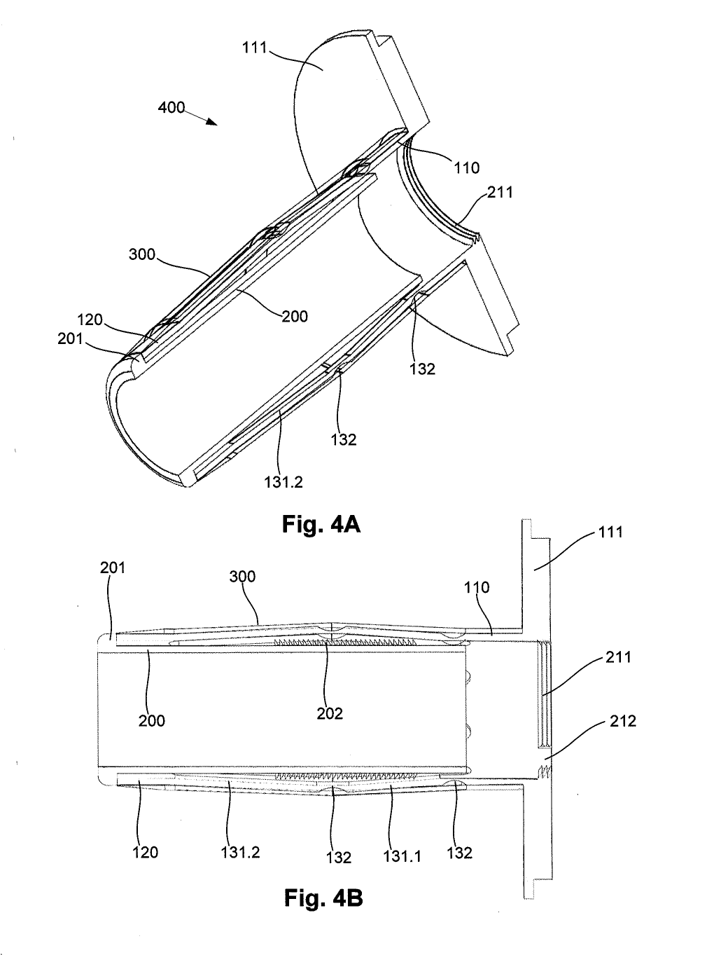

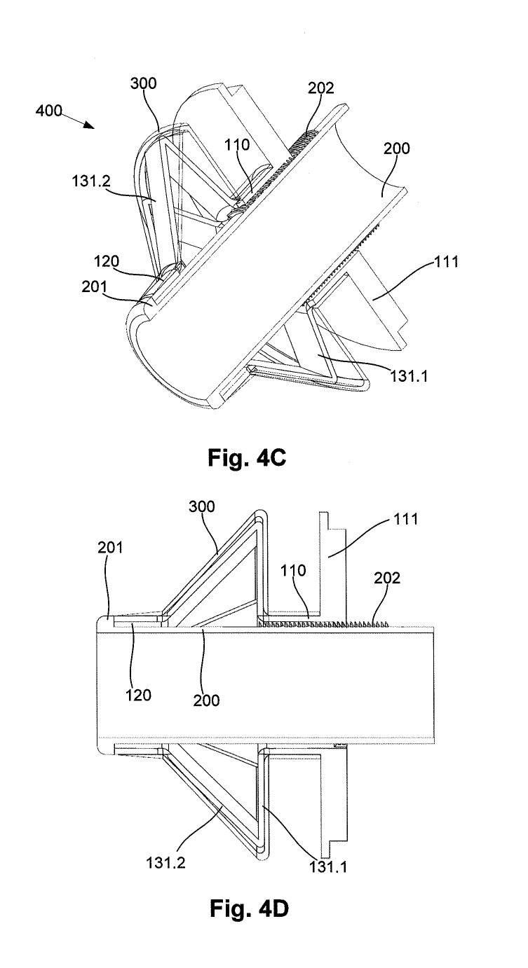

An example of a cannula 400 including the cannula body 100, tubular body 200 and silicone sleeve 300 is shown in FIGS. 4A to 4D.

In particular, as shown in these examples, in the extended configuration, the tubular body 200 is provided inside the cannula body 100 with the shoulder 201 engaging the second end portion 120. The silicone sleeve 300 extends over an outer surface of the cannula body 100, and abuts against the flange 111. When moved into the collapsed configuration, shown in FIGS. 4C and 4D, the tubular body 200 extends through the first end portion 110 allowing the body teeth 202 to engage the cannula teeth 211, thereby locking the cannula body 100 in the collapsed configuration, whilst the silicone sleeve 300 is deformed to accommodate the arms 131, as shown.

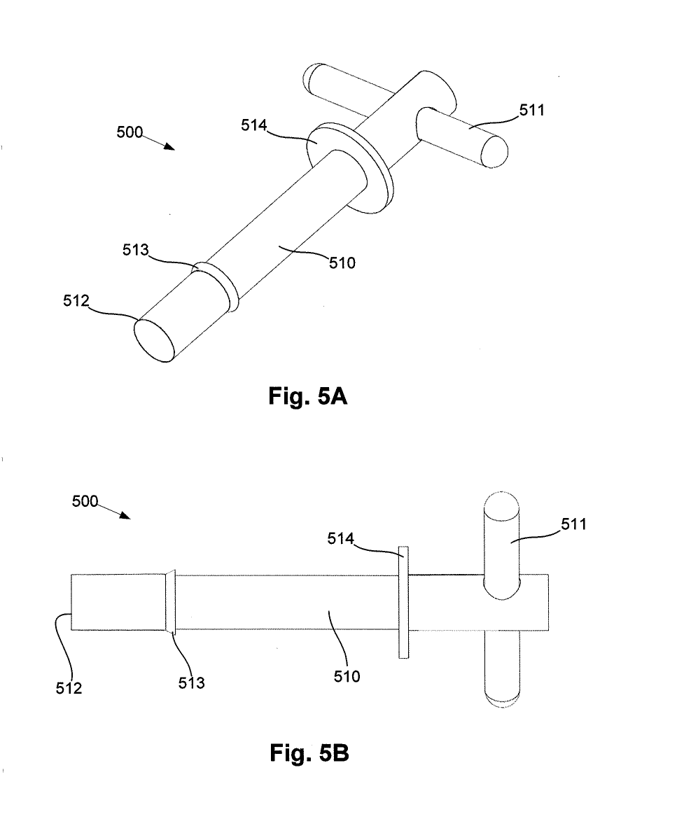

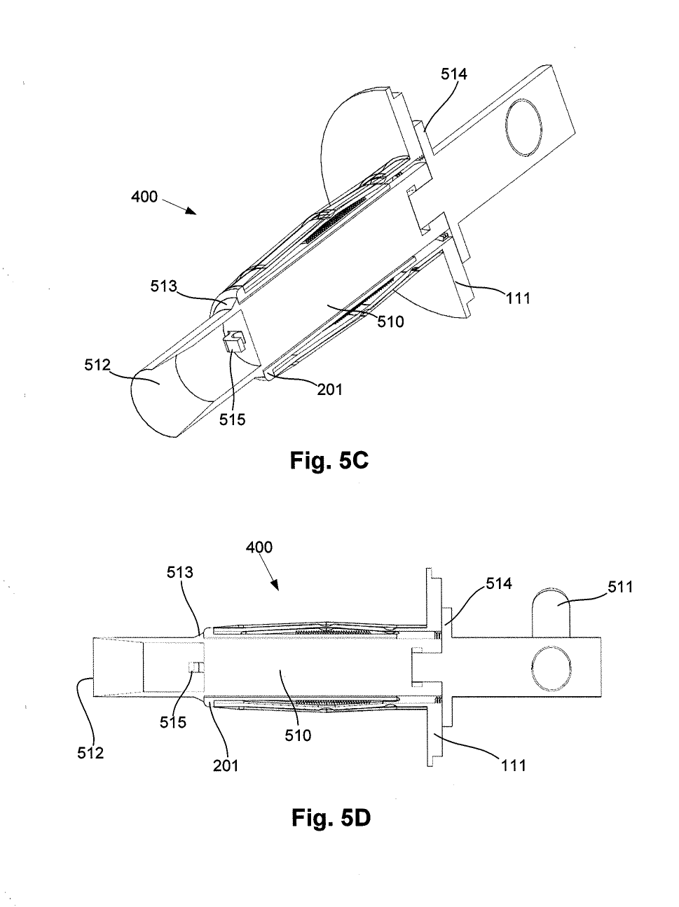

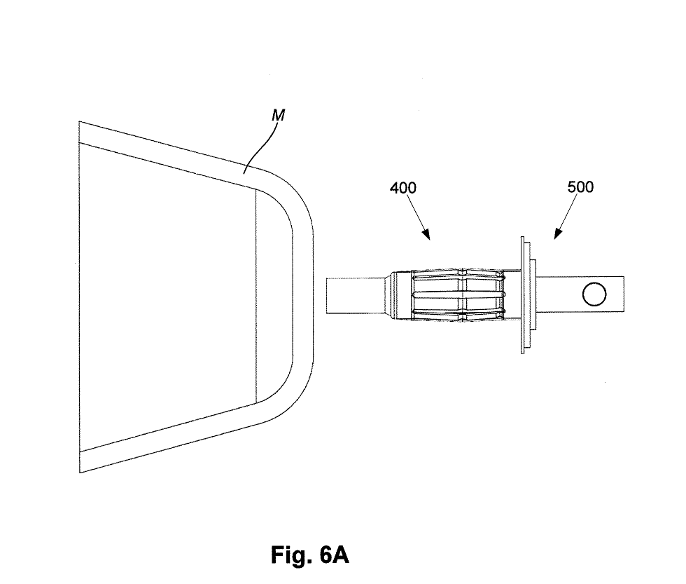

In general, the cannula 400 is inserted into the body utilising a corer, an example of which will now be described in more detail with reference to FIGS. 5A to 5D.

In this example, the corer 500 includes a corer body 510 that extends through and coaxially inwardly of the cannula body 100. A handle 511 is coupled proximate a first end of the corer body 510, allowing a user to hold and manipulate the corer 500 in use, whilst a corer cutting edge 512 is defined at a second opposing end of the corer body 510, for coring the tissue to thereby create an opening therein.

An optional flange 514 can be provided near the handle, which in use abuts against the flange 111, of the cannula body 100, thereby correctly positioning the cannula on the corer 500 and pushing the cannula body 100 into the cored vessel. The corer 500 can also include a hook or screw 515 mounted to the second end of the corer body 510, inwardly of the cutting edge 512, for coupling the corer to removed tissue, as will be described in more detail below.

The corer 500 can also include an engagement mechanism for engaging the cannula, thereby allowing the cannula body 100 to be collapsed in use. In the current example, the engagement mechanism includes a corer shoulder 513 positioned proximate the second end of the corer body 510. In use, the corer is initially inserted into the cannula by inserting the corer body 510 through the tubular body 200 so that the corer shoulder 513 engages an outer surface of the shoulder 201, as shown in FIG. 5C. When the cannula has been provided within the organ, as will be described in more detail below, a user can pull the corer handle 511 away from the flange 111 thereby causing the shoulder 513 to urge the tubular body 200 through the first end portion 110, allowing this to lock as described above.

Once the tubular body 200 is locked in a desired position, the corer can be released and removed from the cannula. This can be achieved either by having the corer shoulder 513 deform upon application of a predetermined force, for example by allowing the corer shoulder 513 collapse, allowing the corer 500 to be withdrawn from the cannula.

Alternatively however, the corer shoulder 513 can extend around only part of the corer body 510, with the tubular body 200 can include a channel extending along an inner surface of the tubular body, which in use can receive the corer shoulder. In this example, relative rotation of the corer 500 and tubular body 200 enable the corer shoulder 513 to be received in the channel, thereby allowing the corer to be withdrawn. The corer shoulder 513 can alternatively be replaced by any suitable engaging mechanism that allows the corer 500 to selectively engage the tubular body 200, and other arrangements using screw threads will be described in more detail below.

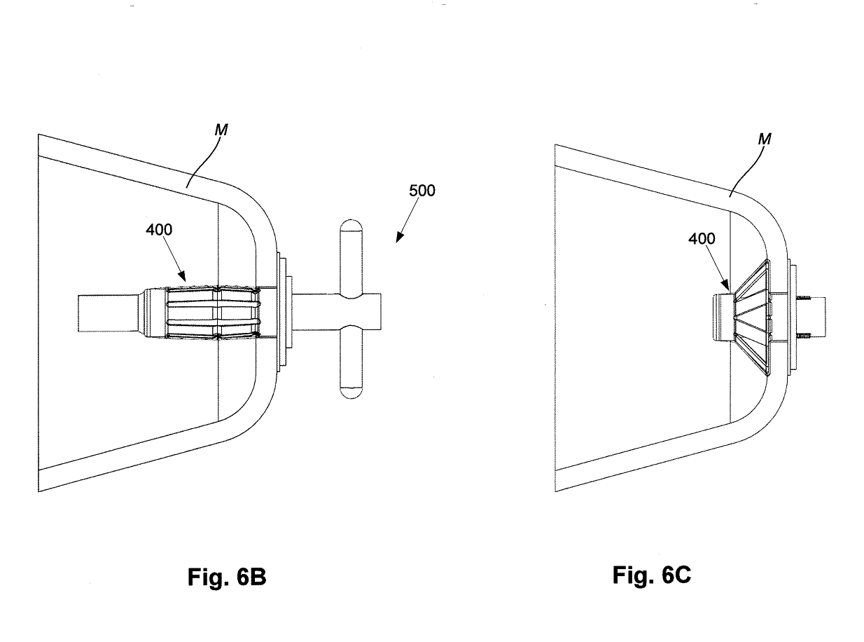

An example of the process of inserting a cannula into an organ such as the heart will now be described with reference to FIGS. 6A to 6C.

In this example, the corer 500 is initially mounted within the cannula 400, as shown. A suture can then be initially inserted into the myocardium with the suture engaging with the hook 515. This allows the section of the myocardium which is removed to remain attached to the corer 500 so that this can be extracted from the heart, when the corer 500 is removed. Alternatively, the hook 515 can be replaced with a screw or spring-like arrangement which engages the myocardium along with the corer 500. The screw or spring-like arrangement would attach to the section of myocardium which is removed as the corer 500 is extracted from the heart.

The corer 500 is then urged against the myocardium M, and optionally reciprocatingly rotated so that the cutting edge 512 cuts through the myocardium M, thereby removing a cylindrical section to provide an opening through the myocardium M. The corer 500 and cannula 400 are then pushed into the opening, until the cannula 400 is fully inserted into the heart, with the flange 111 abutting against an outer surface of the myocardium M.

At this point the corer handle 511 is urged away from the flange 111, urging the cannula into the collapsed configuration, with the myocardium M sandwiched between the flange 111 and the arms 131. In this configuration, the silicone sleeve, and in particular the roughened portion of the silicone sleeve is positioned between the cannula, and in particular the end portion 110 and arms 131, and the myocardium M, thereby encouraging tissue regrowth. The corer 500 is released and removed, so that the cannula 400 provides an opening into the heart. As this occurs, a surgeon would typically seal the opening using their thumb, allowing them to check for unrestricted blood flow, before attaching a connecting tube or the like to the tubular body 200, thereby allowing the cannula to be connected to a device, such as a heart pump, or other device depending on the particular use of the cannula. Alternatively, a valve can be included within the tubular section 200 to prevent blood flow through the cannula until a device, such as a heart pump, is connected. Upon connection of the heart pump, the valve would open, thus allowing blood to flow through the circuit.

Accordingly, the above described arrangement can be inserted into and attached to tissue, such as an organ wall, using a single insertion step. Furthermore, the cannula is generally simpler than previous arrangements, making it cheaper to manufacture and more reliable in use.

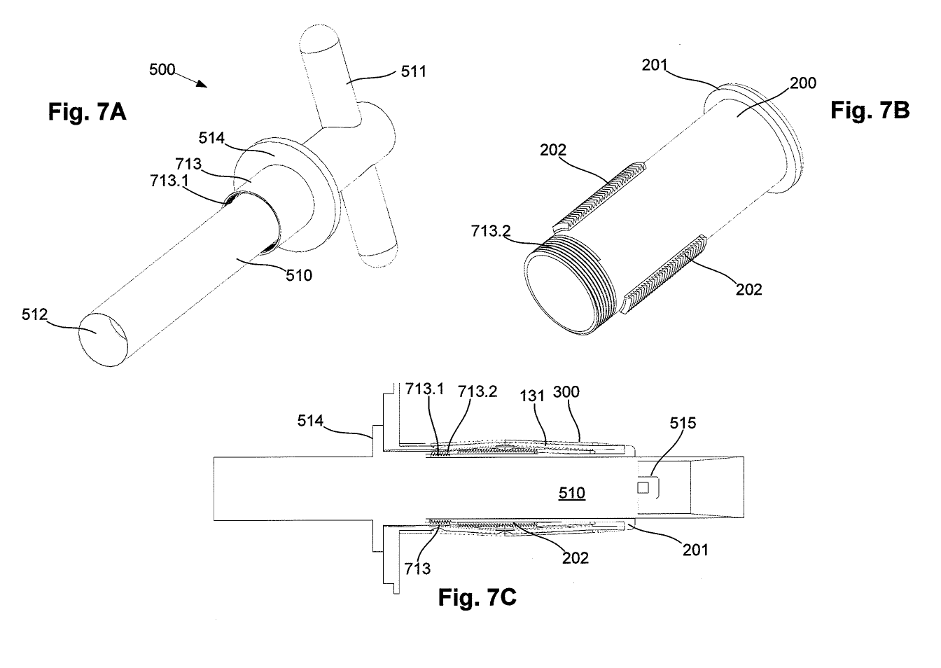

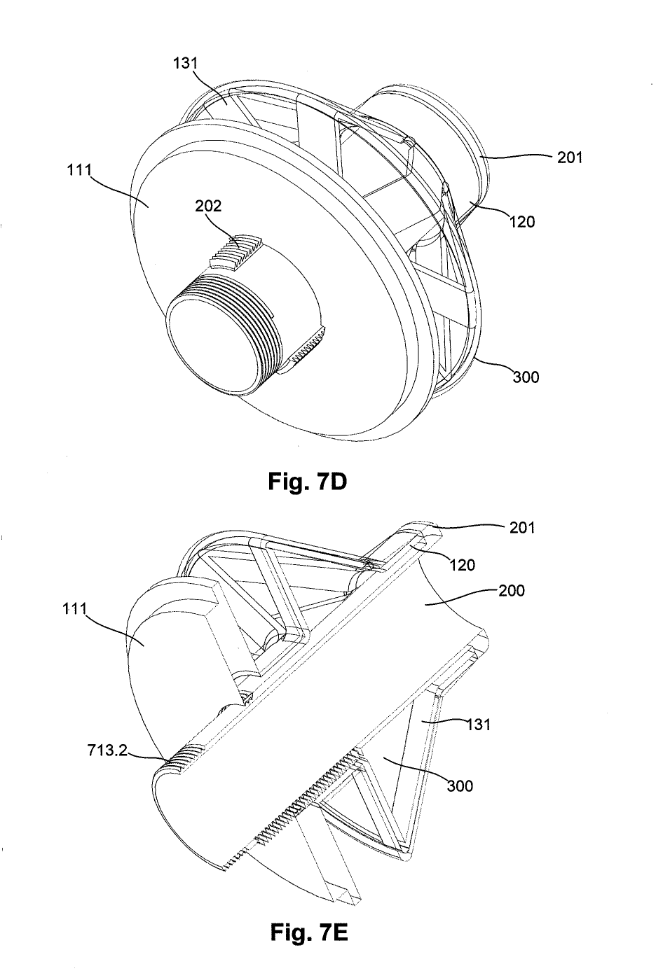

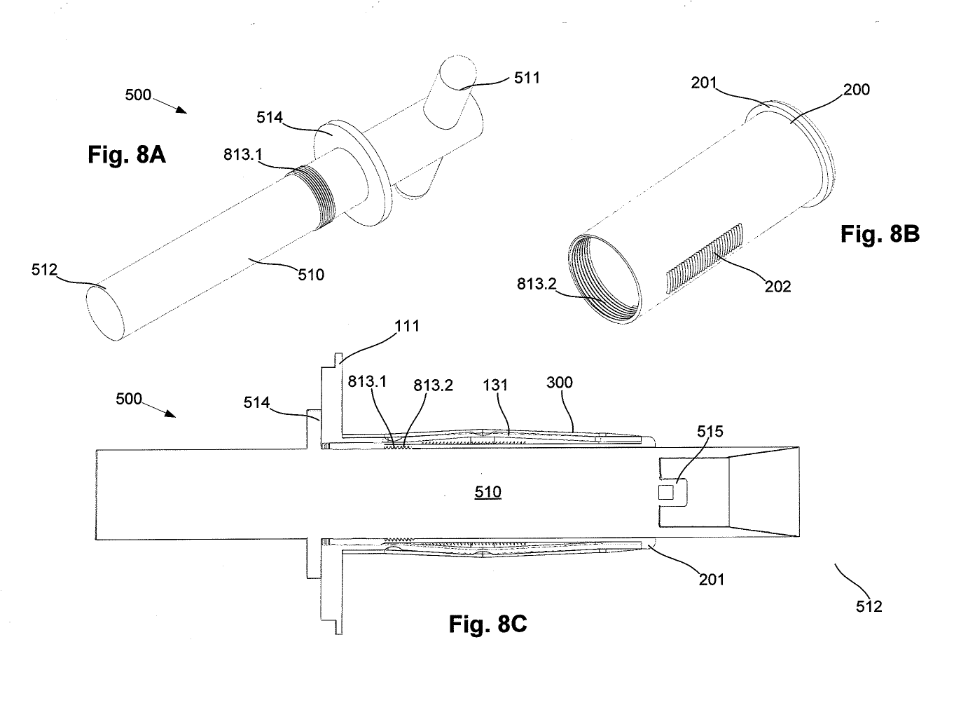

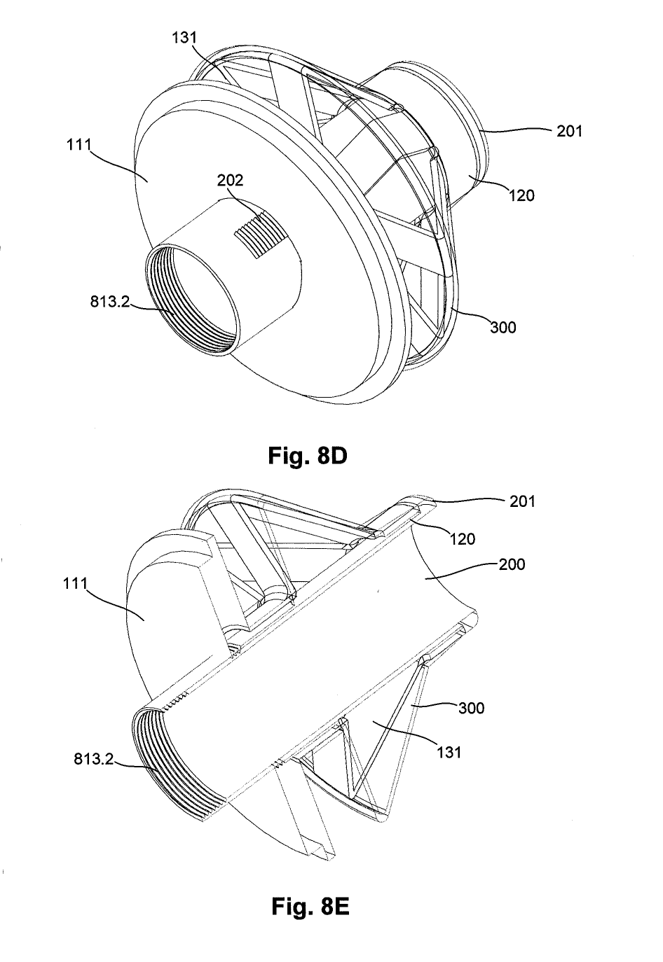

Further examples of cannulae will now be described with reference to FIGS. 7A to 7E and 8A to 8E. For the purpose of these examples, similar reference numerals are used to refer to similar features to the previous examples, and these features will not be described in detail. In these examples, the engagement mechanism for allowing the corer to engage the tubular body is in the form of cooperating screw threads provided on the corer 500 and the tubular body 200, respectively.

In the example of FIGS. 7A to 7E, the corer 500 includes a screw thread support 713 in the form of a tubular body positioned radially outwardly of the corer body 510 and abutting against the flange 514, with a corer screw thread 713.1 being provided on an inner surface of the screw thread body 713. A complementary tube screw thread 713.2 is provided on an outer surface of the tubular body 200 at a second end of the tubular body 200. In use, this allows the tubular body 200 to be attached to the corer 500 by engaging the respective corer and tube screw threads 713.1, 713.2.

A similar arrangement is shown in the example of FIGS. 8A to 8E. However, in this example, the corer screw thread 813.1 is provided on an outer surface of the corer body 510, whilst the tube screw thread 813.2 is provided on an inner surface of the tubular body 200. It will be appreciated that this is therefore largely similar to the example of FIGS. 7A to 7E, albeit without requiring the presence of the tubular with the screw thread support.

In the above examples, in use, the cannula would be supplied with the corer 500 attached to the cannula via the tubular body, as shown in FIGS. 7C and 8C. Following insertion into the tissue wall, the corer 500 is retracted, with the screw threads 713.1, 713.2, 813.1, 813.2 engaging the corer 500 and tubular body 200, so that the cannula collapses into the collapsed position shown in FIGS. 7D, 7E, 8D and 8E, with the body teeth 202 and the cannula teeth 211 engaging to lock the tubular body 200 relative to the first end portion 110, with the tissue wall held between the flange 111 and arms 131, as described with respect to the previous examples. Following this, rotation of the corer 500 can be used to undo the complimentary screw threads 713.1, 713.2, 813.1, 813.2, thereby decoupling the corer 500 from the cannula, allowing the corer 500 to be removed, as described in the previous examples.

Once removed, the tube screw threads 713.2, 813.2 can advantageously be used to allow a device, such as a heart pump or the like to be connected thereto.

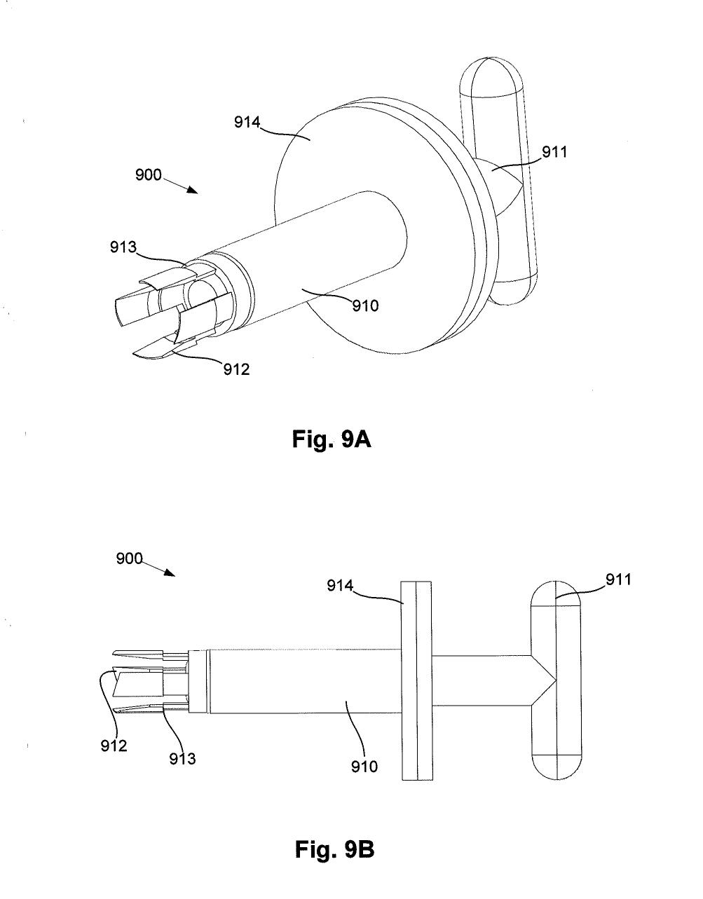

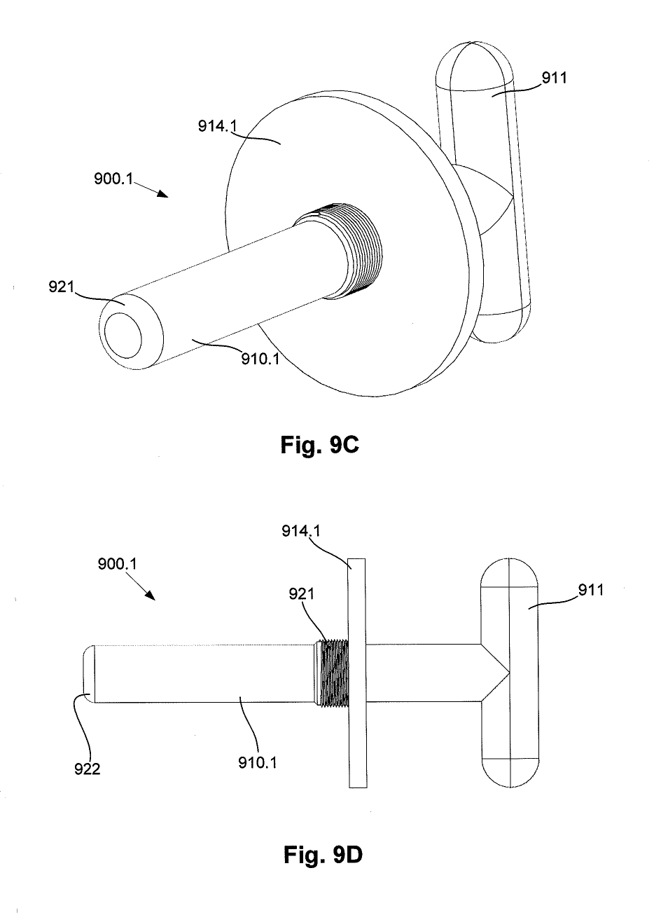

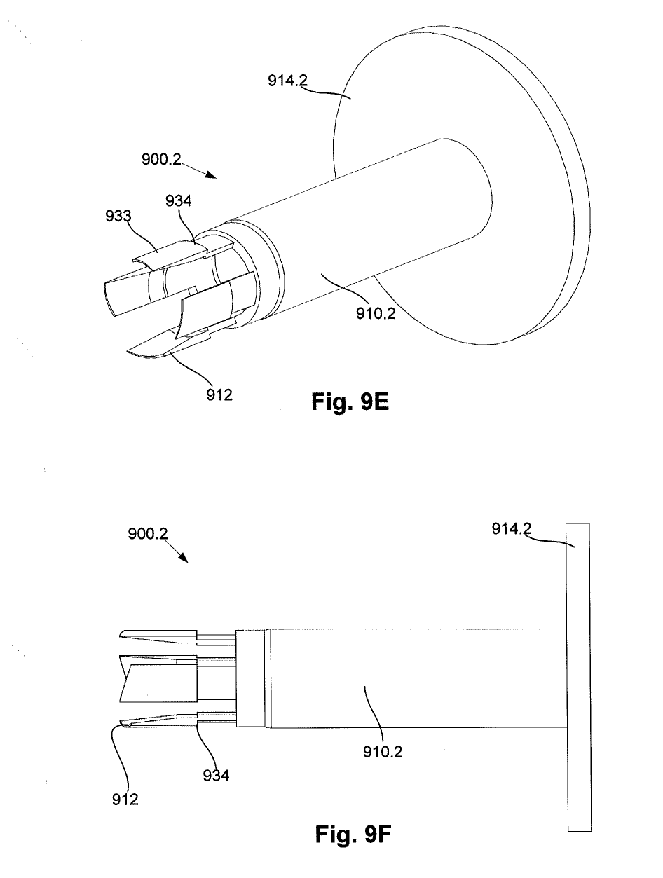

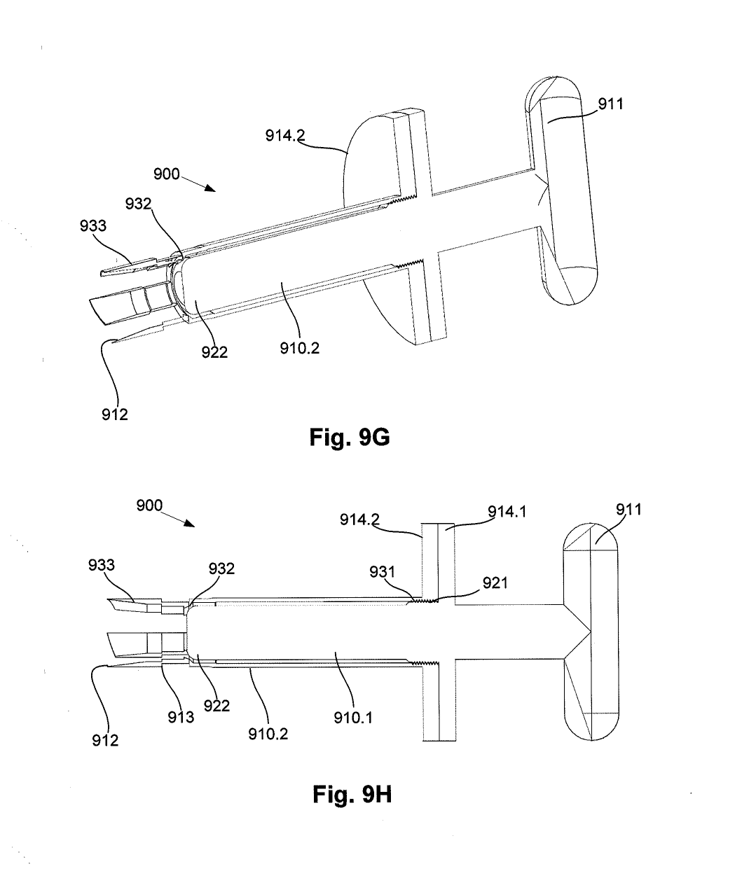

A further example of a corer will now be described in more detail with reference to FIGS. 9A to 9H.

In this example, the corer 900 includes a corer body 910 that extends through and coaxially inwardly of the cannula body 100. A handle 911 is coupled proximate a first end of the corer body 910, allowing a user to hold and manipulate the corer 900 in use, whilst corer cutting edges 912 are defined at a second opposing end of the corer body 910, for coring the tissue to thereby create an opening therein. An optional flange 914 can be provided near the handle, which in use abuts against the flange 111, of the cannula body 100, thereby correctly positioning the cannula on the corer 900 and pushing the cannula body 100 into the cored tissue. It will be appreciated from this that the corer 900 is generally similar to the corer 500 previously described.

However, in this example, the corer 900 is formed from first and second corer bodies 900.1, 900.2. The first corer body 900.1 includes a handle 911 coupled to a first end of an elongate cylindrical shaft 910.1, which terminates in a chamfered second end 922. The first corer body 900.1 includes a first flange portion 914.1 mounted part way along the shaft 910.1, with an external screw thread 921 positioned on the shaft 910.1 adjacent the first flange portion 914.1.

The second corer body 900.2 includes an elongate cylindrical hollow tube 910.2 having a second flange portion 914.2 at a first end, and terminating in a number of circumferentially spaced fingers 933 at a second end. An internal screw thread 931 is positioned on an inner surface of the tube 910.2 adjacent the second flange portion 914.2, with the tube 910.2 being sized to allow it to be positioned over the shaft 910.1, and threadingly engaged with the screw thread 921 on the shaft 910.1.

The fingers 933 are coupled to a first end to the tube 910.2, whilst the second end forms the corer cutting edges. An outer surface of the fingers 933 includes a step 934 extending circumferentially around an outer surface mid-way along the fingers 933. A shoulder 932 is defined circumferentially around an inner surface of the finders 933, proximate the first end. In use, when the shaft 910.1 of the first corer body 900.1 is inserted into the tube 910.2 of the second corer body 900.2, the chamfered second end 922 engages the shoulder 932. This action urges the fingers 933 apart, so that the step 934 engages an outer surface of the shoulder 210 of the tubular body 200, thereby acting as an engagement mechanism for engaging the cannula as previously described. Additionally, this more closely aligns the cutting surfaces 912 with an outer surface of the cannula, ensuring the diameter of the opening created by the corer more closely matches the diameter of the cannula, thereby facilitating cannula insertion.

Accordingly, in use, the second corer body 900.2 and first corer body 900.1 are inserted into the tubular body 200, before being threadingly engaged, to thereby urge the shaft 910.1 along the tube 910.2, and thereby activate the engagement mechanism, by urging the fingers 933 apart, so the step 934 engages the tubular body 200. Following this, the corer is used to create the tissue opening, allowing the cannula to be inserted therethrough. Once appropriately position, the user can pull the corer handle 911 away from the cannula flange 111 thereby causing the step 934 to urge the tubular body 200 through the first end portion 110, allowing this to lock as described above. Following this the first and second corer bodies can be unscrewed from each other, releasing the fingers 933 and allowing the corer 900 to be extracted.

Accordingly, in this example, the corer includes first and second coaxially arranged corer bodies 900.1, 900.2. Relative axial movement of the corer bodies 900.1, 900.2, for example through threading engagement of the corer bodies 900.1, 900.2 urges fingers 933 of the second corer body outwardly to thereby engage the tubular body. The fingers 933 also define corer cutting edges.

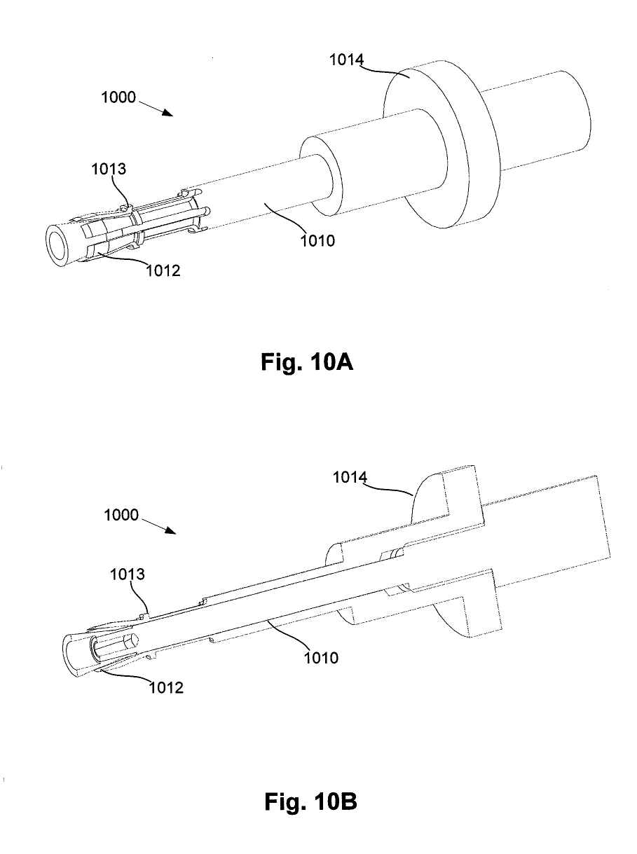

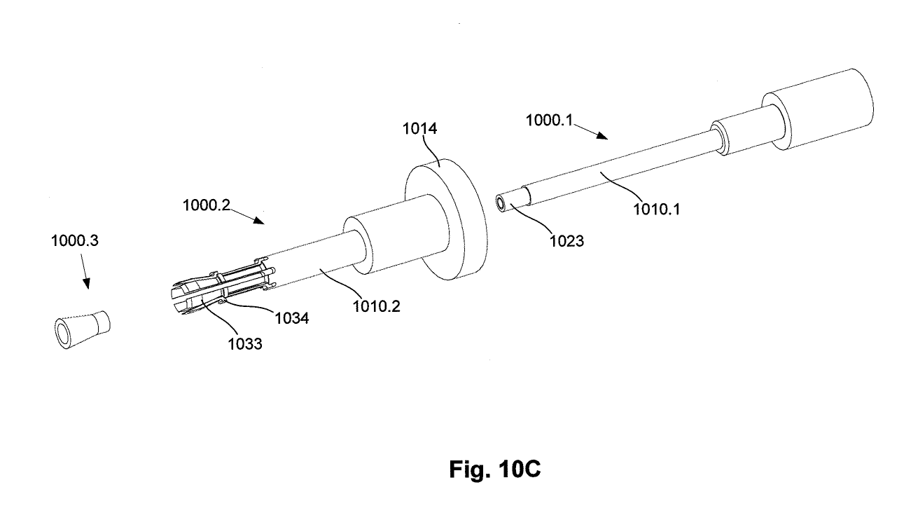

A further example of a corer will now be described in more detail with reference to FIGS. 10A to 10C.

In this example, the corer 1000 includes a corer body 1010 that extends through and coaxially inwardly of the cannula body 100. A handle (not shown) is coupled proximate a first end of the corer body 1010, allowing a user to hold and manipulate the corer 1000 in use, whilst corer cutting edges 1012 are defined at a second opposing end of the corer body 1010, for coring the tissue to thereby create an opening therein. An optional flange 1014 can be provided near the handle, which in use abuts against the flange 111, of the cannula body 100, thereby correctly positioning the cannula on the corer 1000 and pushing the cannula body 100 into the cored tissue. It will be appreciated from this that the corer 1000 is generally similar to the corer 900 previously described.

However, in this example, the corer 1000 is formed from first and second corer bodies 1000.1, 1000.2 and an end piece 1000.3. The first corer body 1000.1 includes the handle (not shown) coupled to a first end of an elongate cylindrical shaft 1010.1, which terminates in a mounting 1023. The second corer body 1000.2 includes an elongate cylindrical hollow tube 1010.2 having the flange 1014 at a first end, and terminating in a number of circumferentially spaced fingers 1033 at a second end. The tube 910.2 is sized to allow it to be positioned over the shaft 1010.1. The end piece 1000.3 is frustoconically shaped and can be coupled to the mounting 1023 of the first corer body 1000.1 once the second corer body 1000.2 is positioned thereon, so that the end piece defines a flared end. In use, relative axial movement of the first and second corer bodies 1000.1, 1000.2 causes the end piece 1000.3 to urge the fingers 1033 apart, so that the step 1034 engages an outer surface of the shoulder 210 of the tubular body 200, thereby acting as an engagement mechanism for engaging the cannula as previously described. Again, this more closely aligns the cutting surfaces 1012 with an outer surface of the cannula, ensuring the diameter of the opening created by the corer more closely matches the diameter of the cannula, thereby facilitating cannula insertion.

Accordingly, in use, the second corer body 900.2 and first corer body 900.1 are inserted into the tubular body 200, before being threadingly engaged, to thereby urge the shaft 910.1 along the tube 910.2, and thereby activate the engagement mechanism, by urging the fingers 933 apart, so the step 934 engages the tubular body 200.

Accordingly, it will be appreciated that this can function in a manner similar to the corer 900 described above and this will not therefore be described in further detail.

The above described cannula and insertion method finds particular application in providing an opening through tissue in a biological subject, such as a wall of an organ. However, the use of the cannula is not so limited, and could be used in any circumstance in which it is desired to insert an opening in a body or other member. Thus, for example, this could be used to insert an opening into the wall of a fluid pipe or the like. It will therefore be appreciated that specific reference to tissue of a biological subject is not intended to be either exclusive or limiting, although this is a preferred use.

The term biological subject is also not intended to be limiting and could include any suitable subject including, but not restricted to, humans as well as non-human animals including primates, livestock animals (e.g., sheep, cows, horses, donkeys, pigs), laboratory test animals (e.g., rabbits, mice, rats, guinea pigs, hamsters), companion animals (e.g., cats, dogs) and captive wild animals (e.g., foxes, deer, dingoes).

Throughout this specification and claims which follow, unless the context requires otherwise, the word “comprise”, and variations such as “comprises” or “comprising”, will be understood to imply the inclusion of a stated integer or group of integers or steps but not the exclusion of any other integer or group of integers.

Persons skilled in the art will appreciate that numerous variations and modifications will become apparent. All such variations and modifications which become apparent to persons skilled in the art, should be considered to fall within the spirit and scope that the invention broadly appearing before described.

1. A cannula including a hollow cannula body having:
a) first and second tubular end portions;
b) a collapsible section interconnecting the end portions, the collapsible section including a plurality of circumferentially spaced arms extending between the end portions, wherein in an extended configuration the arms are substantially aligned with the first and second end portions and in a collapsed configuration the arms deform to extend radially outwardly;
c) a flange extending radially outwardly from the first end portion, so that the arms and flange are spaced apart when the cannula body is in the collapsed configuration, thereby allowing tissue to be sandwiched therebetween to thereby effect a seal between the cannula and the tissue so that the cannula provides an opening through the tissue.

2. A cannula according to claim 1, wherein the arms include deformable portions that cause the arms to deform in predefined positions when collapsing.

3. A cannula according to claim 1, wherein in the collapsed configuration, the arms extend outwardly in a generally “V” shaped arrangement.

4. A cannula according to claim 1, wherein the arms include a first arm portion coupled to the first end portion and a second arm portion coupled to the second end portion, the first and second arm portions being interconnected and wherein the first arm portion is shorter than the second arm portion so that the first arm portion is substantially parallel to the flange when the cannula body is in the collapsed configuration.

5. A cannula according to claim 1, wherein the cannula includes a locking mechanism for locking the cannula in the collapsed configuration.

6. A cannula according to claim 5, wherein the locking mechanism is adjustable to allow the relative separation of the flange and arms to be adjusted.

7. A cannula according to claim 5, wherein the locking mechanism interconnects the first and second end portions.

8. A cannula according to claim 1, wherein the cannula includes a tubular body extending through and coaxially inwardly of the cannula body.

9. A cannula according to claim 8, wherein the tubular body includes a shoulder at a first end for engaging the second end portion.

10. A cannula according to claim 8, wherein the tubular body includes a number of axially spaced body teeth extending circumferentially around at least part of an outer surface of the tubular body and wherein the first end portion includes a number of axially spaced cannula teeth extending circumferentially around at least part of an inner surface of the first end portion, the body teeth and cannula teeth selectively engaging to thereby lock the tubular body relative to the first end portion.

11. A cannula according to claim 10, wherein relative rotation of the tubular body and first end portion allow the body teeth and cannula teeth to selectively disengage.

12. A cannula according to claim 1, wherein the cannula includes a sleeve extending over and coaxially outwardly of at least part of the cannula body.

13. A cannula according to claim 12, wherein the sleeve is a silicone sleeve.

14. A cannula according to claim 12, wherein the sleeve has a low friction outer surface to facilitate insertion of the cannula into the tissue.

15. A cannula according to claim 12, wherein the sleeve includes a roughened outer surface at least proximate an end of the sleeve adjacent the flange.

16. A cannula according to claim 1, wherein the flange includes a felt surface that in use engages the tissue surface to thereby seal the cannula against the tissue.

17. A cannula system including:
a) a cannula including a hollow cannula body having:
i) first and second tubular end portions;
ii) a collapsible section interconnecting the end portions, the collapsible section including a plurality of circumferentially spaced arms extending between the end portions, wherein in an extended configuration the arms are substantially aligned with the first and second end portions and in a collapsed configuration the arms deform to extend radially outwardly;
iii) a flange extending radially outwardly from the first end portion, so that the arms and flange are spaced apart when the cannula body is in the collapsed configuration, thereby allowing tissue to be sandwiched therebetween to thereby effect a seal between the cannula and the tissue so that the cannula provides an opening through the tissue; and,
b) a corer including:
i) a corer body that in use extends through and coaxially inwardly of the cannula body;
ii) a handle proximate a first end of the corer body for manipulating the corer;
iii) a corer cutting edge at a second opposing end of the corer body for coring the tissue to thereby creating an opening in the tissue; and,
iv) an engagement mechanism for selectively engaging the cannula, thereby allowing the cannula body to be collapsed in use.

18. A cannula system according to claim 17, wherein the cannula includes a tubular body extending through and coaxially inwardly of the cannula body, the corer extending through and coaxially inwardly of the tubular body.

19. A cannula system according to claim 18, wherein the engagement mechanism includes a corer shoulder that engages the tubular body.

20. A cannula system according to claim 18, wherein the engagement mechanism includes a corer threaded portion that selectively couples to a corresponding tubular body threaded portion upon relative rotation of the corer and tubular body.

21. A cannula system according to claim 20, wherein the corer threaded portion is provided on an outer surface of the corer body and the tubular body threaded portion is provided on an inner surface of the tubular body.

22. A cannula system according to claim 18, wherein the corer includes first and second coaxially arranged corer bodies, and wherein relative axial movement of the corer bodies urges fingers of the second corer body outwardly to thereby engage the tubular body.

23. A cannula system according to claim 22, wherein the fingers define corer cutting edges.

24. A cannula system according to claim 22, wherein the first and second corer bodies are threadingly engaged to cause relative axial movement of the corer bodies.

25. A cannula system according to claim 17, wherein the corer includes at least one of:
a) a hook that in use is coupled to a suture passing through the tissue to be removed; and,
b) a tissue screw that in use engages the tissue to be removed.

26. A method of inserting a cannula mounted on a corer, wherein:
a) the cannula includes a cannula body having:
i) first and second tubular end portions;
ii) a collapsible section interconnecting the end portions, the collapsible section including arms extending between the end portions;
iii) a flange extending radially outwardly from the first end portion; and,
b) the corer includes:
i) a corer body that in use extends through and coaxially inwardly of the cannula body;
ii) a handle for manipulating the corer;
iii) a corer cutting edge; and,
iv) an engagement mechanism, wherein the method includes:
(1) inserting the corer cutting edge through the tissue to thereby create an opening in the tissue;
(2) urging the cannula through the opening in the tissue, with the cannula in an extended configuration in which the arms are substantially aligned with the first and second end portions;
(3) urging the corer towards the first end portion so that the engagement mechanism urges the second end portion towards the first end portion to thereby move the cannula to a collapsed configuration in which the arms deform to extend radially outwardly so that the arms and flange are spaced apart thereby allowing tissue to be sandwiched therebetween to thereby secure the cannula to the tissue; and,
(4) withdrawing the corer from the cannula so that the cannula provides an opening through the tissue.

27. A method according to claim 26, wherein the method includes using a locking mechanism to lock the cannula in the collapsed configuration.

28. A method according to claim 26, wherein the cannula includes a tubular body extending through and coaxially inwardly of the cannula body and wherein the method includes rotating a tubular body relative to the cannula body to thereby selectively engage the locking mechanism.

29. A method according to claim 26, wherein the cannula includes a tubular body extending through and coaxially inwardly of the cannula body, wherein the engagement mechanism includes a corer threaded portion that selectively couples to a corresponding tubular body threaded portion and wherein the method includes rotating the corer relative to the tubular body thereby allowing the corer to be withdrawn from the cannula.
